(19) United States
(12) Patent Application Publication
(10) Pub. No.: US 2016/0166625 A1
(43) Pub. Date: Jun. 16, 2016

(54) PHARMACEUTICAL METHOD FOR CANNABIS DISTRIBUTION AND AN AILMENT-TARGETED CANNABIS COMPOSITION

(71) Applicant: CANNA PURE INC., Boca Raton, FL (US)

(72) Inventor: Herbert F. Postma, Boca Raton, FL (US)

(21) Appl. No.: 14/966,924

(22) Filed: Dec. 11, 2015

Related U.S. Application Data
(60) Provisional application No. 62/090,445, filed on Dec. 11, 2014.

(51) Int. Cl. A61K 36/185; G07F 13/10; G06F 19/00; G07F 13/08

(52) U.S. Cl. CPC A61K 36/185; G07F 13/08; G07F 13/10; G06F 19/3462

(57) ABSTRACT

A method of dispensing cannabis is disclosed that includes providing a cannabis composition via a dispensary, the cannabis composition having an effective amount of cannabis; formulated as an ailment-targeted treatment; and formed so as to deliver the effective amount of cannabis to a subject through a delivery method other than inhalation or consumption of an edible food product.

### CROSS-REFERENCE TO RELATED APPLICATION

This application claims priority to U.S. Provisional Patent Application No. 62/090,445 filed Dec. 11, 2014, the entirety of which is incorporated by reference.

### FIELD OF THE INVENTION

The present invention relates generally to a system and method for cannabis distribution, and more particularly, relates to an ailment-targeted cannabis composition including an effective amount of cannabis.

### BACKGROUND OF THE INVENTION

It is well known that cannabis is typically distributed as an herbal product that is either inhaled through smoking or vaporizing or is included as an ingredient within an edible food product, such as brownies or other confections. Cannabis has recently been declared a legal compound in several U.S. states, yet cannabis still carries a negative association in the minds of many consumers and the general public. Therefore, what is needed is a method of providing cannabis in a more discrete form, using more conventional medical delivery systems, and distributed as part of a dispensary business model that more closely mirrors other consumer-accepted pharmaceutical dispensary business models having a more positive association in the minds of the general public.

Therefore, a need exists to overcome the problems with the prior art as discussed above.

### SUMMARY OF THE INVENTION

The invention provides a pharmaceutical method for cannabis distribution and an ailment-targeted cannabis composition that overcomes the hereinafore-mentioned disadvantages of the heretofore-known devices and methods of this general type.

With the foregoing and other objects in view, there is provided, in accordance with the invention, a method of dispensing cannabis, the method including providing a cannabis composition via a dispensary, the cannabis composition having an effective amount of cannabis; formulated as an ailment-targeted treatment; and formed so as to deliver the effective amount of cannabis to a subject through a delivery method other than inhalation or consumption of an edible food product.

In accordance with another feature, an embodiment of the present invention includes a cannabis composition comprising an effective amount of cannabis configured for treatment of a singular specific ailment; and formed so as to deliver the effective amount of cannabis to a subject through a delivery method other than inhalation or consumption of an edible food product.

Although the invention is illustrated and described herein as embodied in a pharmaceutical method for cannabis distribution and an ailment-targeted cannabis composition, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. In this document, the term “longitudinal” should be understood to mean in a direction corresponding to an elongated direction of the dispensary. The terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a schematic view of a flow chart of an exemplary method of dispensing cannabis, in accordance with the present invention;

FIG. 2 is a schematic view of a block diagram of an exemplary embodiment of a dispensary, in accordance with the present invention; and

FIG. 3 is a block diagram of a data processing system that may be implemented as a network device in accordance with an embodiment of the present invention.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

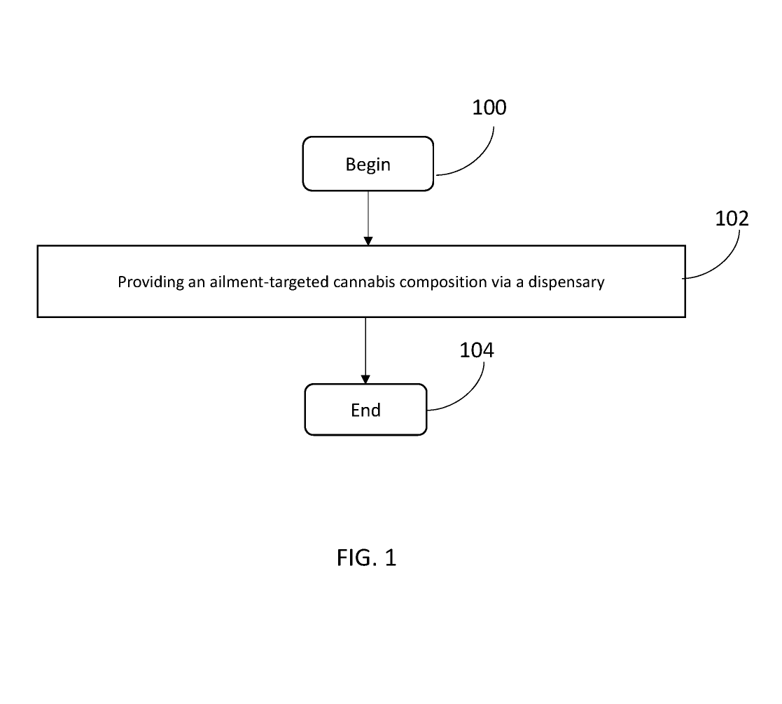

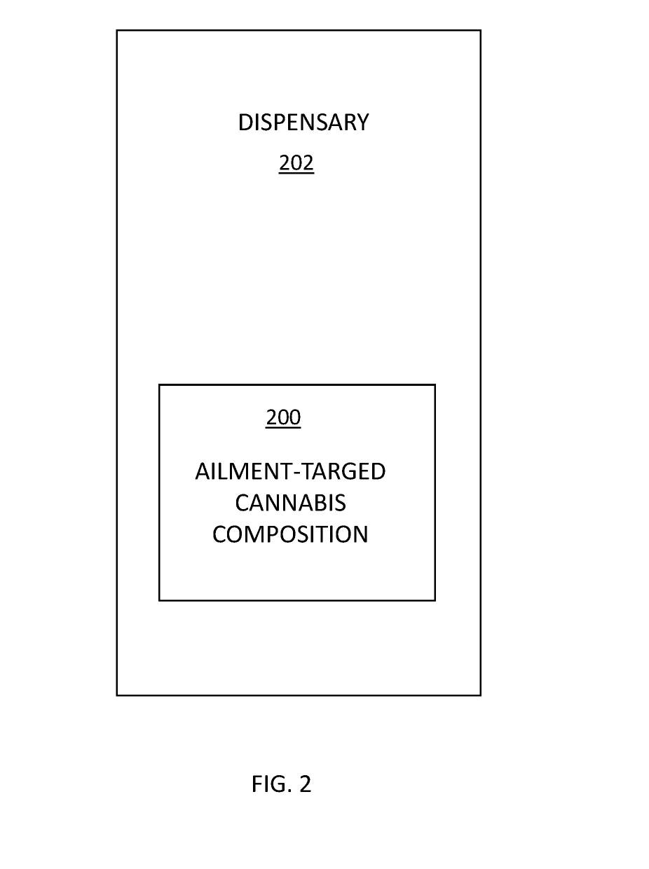

Referring now to FIGS. 1 and 2, one embodiment of the present invention is shown in schematic views. FIGS. 1 and 2 show several advantageous features of the present invention, but, as will be described below, the invention can be provided in several shapes, sizes, combinations of features and components, and varying numbers and functions of the components. The first example of an exemplary method of dispensing cannabis, as shown in FIG. 1, and described as follows with reference to FIG. 2, begins at step 100 and immediately proceeds to step 102, where an ailment-targeted cannabis composition 200 is provided via a dispensary 202.

Although FIG. 1 shows a specific order of executing the process steps, the order of executing the steps may be changed relative to the order shown in certain embodiments. Also, two or more blocks shown in succession may be executed concurrently or with partial concurrence in some embodiments. Certain steps may also be omitted in FIG. 1 for the sake of brevity. In some embodiments, some or all of the process steps included in FIG. 1 can be combined into a single process.

In one embodiment, the dispensary 202 is a traditional pharmacy, such as a drug store or a pharmacy located in a grocery outlet. In another embodiment, the dispensary 202 is a retail store. In yet another embodiment, the dispensary 202 is a kiosk. In yet another embodiment, the dispensary 202 is a virtual dispensary 202 in the form of a web site where consumers can submit orders for and request shipping of the cannabis composition 200 without having to visit a physical location. In one embodiment, the website can provide directions to dispensaries selected by the consumer or located proximate to an area in which the consumer resides. As used herein, the term “dispensary” is intended to indicate an area, building, structure, or website or software application portal where consumers can order and receive prescription cannabis compositions 200. In some embodiments, the dispensary 202 will employ cannabis professionals, such as pharmacists, physicians, or other certified experts that can assist consumers with ordering and receiving prescription cannabis compositions 200, instructing consumers on how to use their particular prescription cannabis compositions 200, and verifying correct dosages of the cannabis composition 200.

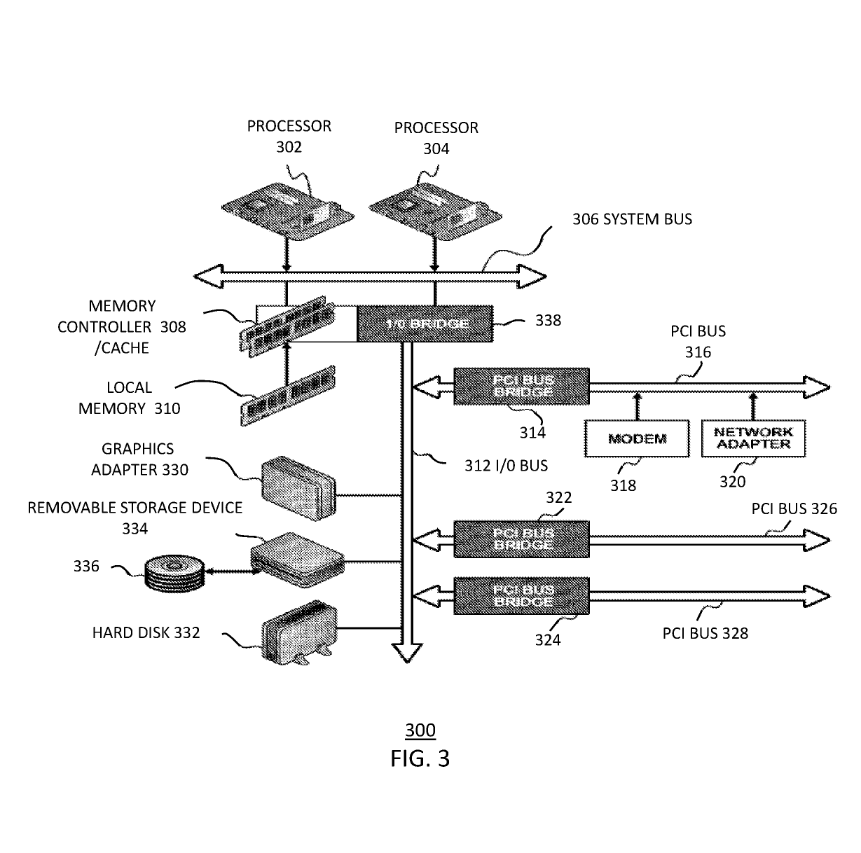

Referring now briefly to FIG. 3, a block diagram of a data processing system 300 that may be implemented as a server, such as the server for a website dispensary 202, or implemented as a personal computer, mobile electronic device, recording device, or other computing device coupled to the Internet for processing cannabis composition 200 orders for consumers, in accordance with one embodiment of the present invention. The data processing system 300 may be a symmetric multiprocessor (SMP) system including a plurality of processors 302 and 304 connected to system bus 306. Alternatively, a single processor system may be employed. Also, connected to system bus 306 is memory controller/cache 308, which provides an interface to local memory 310. An I/O bus bridge 338 is connected to system bus 306 and provides an interface to I/O bus 312. The memory controller/cache 308 and I/O bus bridge 338 may be integrated as depicted. The processor 302 or 304 in conjunction with memory controller 308 controls what data is stored in memory 310. The processor 302 and/or 304 and memory controller 308 can serve as a data counter for counting the rate of data flow to the memory 310 or from the memory 310 and can also count the total volume of data accessed to or from the memory 310. The processor 302 or 304 can also work in conjunction with any other memory device or storage location.

Peripheral component interconnect (PCI) bus bridge 314 connected to I/O bus 312 provides an interface to PCI local bus 316. A number of modems 318, or wireless cards, may be connected to PCI bus 316. Typical PCI bus implementations will support four PCI expansion slots or add-in connectors. PCI includes, but is not necessarily limited to, PCI-X and PCI Express components. Communications links to the network of computers communicatively coupled to the Internet may be provided through the modem 318 and network adapter 320 connected to PCI local bus 316 through add-in boards.

Additional PCI bus bridges 322 and 324 provide interfaces for additional PCI buses 326 and 328, from which additional modems or network adapters may be supported. In this manner, the data processing system 300 allows connections to a multiple network of computers. A graphics adapter 330 and hard disk 332 may also be connected to I/O bus 312 as depicted, either directly or indirectly.

Those of ordinary skill in the art will appreciate that the hardware depicted in FIG. 3 may vary. For example, other peripheral devices, such as optical disk drives and the like, also may be used in addition to or in place of the hardware depicted. The depicted example is not meant to imply architectural limitations with respect to the present invention.

The processes explained in detail above can be embodied in a computer program. Computer programs (also called computer control logic) are stored in memory such as main memory 310, removable storage drive 334, removable media 336, hard disk 332, and signals. Such computer programs, when executed, enable the computer system to perform the features of the present invention as discussed herein. In particular, the computer programs, when executed, cause the processor 302 and/or 304 to perform the steps of some embodiments of the present invention.

In this document, the terms “computer program medium,” “computer usable medium,” and “computer readable medium” are used to generally refer to media such as main memory 310, removable storage drive 334, removable media 336, hard disk 332, and signals. These computer program products are means for providing software to the computer system. The computer readable medium allows the computer system to read data, instructions, messages or message packets, and other computer readable information from the computer readable medium. The computer readable medium, for example, may include non-volatile memory, such as Floppy, ROM, Flash memory, Disk drive memory, CD-ROM, and other permanent storage. It is useful, for example, for transporting information, such as data and computer/programming instructions, between computer systems. Furthermore, the computer readable medium may comprise computer readable information in a transitory state medium such as a network link and/or a network interface, including a wired or wireless network that allows a computer to read such computer readable information.

Referring again to FIGS. 1 and 2, in one embodiment, the cannabis composition 200 includes an effective amount of cannabis. As used herein, the term “effective amount” is intended to indicate a dosage of cannabis effective to treat a targeted ailment. In one embodiment, the effective amount is the prescribed dosage for the particular consumer. In another embodiment, the effective amount is an over-the-counter suggested dosage for a consumer of a particular demographic, such as age, weight, sex, or targeted ailment. The cannabis can be provided in many forms, such as, for example, a ground herbal form, an extract from a cannabis plant, or other product or derivative of a cannabis plant. In some embodiments, the cannabis composition 200 can include additional compounds or substances, such as pharmaceutically acceptable carriers, excipients, diluents, flavoring, coloring, or other active ingredients that also aid in the treatment of the targeted ailment or associated side effects. In one embodiment, the cannabis is organic. In another embodiment, the cannabis can be considered a medical-grade cannabis.

In one embodiment, the cannabis composition 200 is formulated as an ailment-targeted treatment. As used herein, the term “ailment-targeted treatment” is intended to indicate that the cannabis composition 200 is especially formulated by medical professionals to treat a targeted ailment. For example, the amount of cannabis, the form of the cannabis composition 200, the type of cannabis plant used, and/or the processing of the cannabis plant or other derivative can be altered and tailored according to a specific ailment. In one embodiment, the ailment is pain. In another embodiment, the ailment is insomnia. In another embodiment, the ailment is depression. In yet other embodiments, the ailment can be, for example, Crohn's disease, colitis, anxiety, post-traumatic stress disorder (PTSD), seizures, glaucoma, cancer, side effects from cancer treatment, bulimia, anorexia, obesity, and the like. Advantageously, the present invention allows cannabis to be provided to consumers and patients specially formulated to treat the patient's specific ailment, rather than receiving a generic form and amount of cannabis not tailored for a specific treatment. This also enhances the consumer experience by providing alternative cannabis delivery forms that do not possess the negative stigma associated with cannabis use.

In one embodiment, the cannabis composition 200 is formed so as to deliver the effective amount of cannabis to a subject through a delivery method other than inhalation or consumption of an edible food product. In one embodiment, the subject is a human adult. In another embodiment, the subject is a human child. In yet another embodiment, the subject is a non-human animal, such as a pet. In another embodiment the delivery method includes a capsule. In one embodiment, the capsule provides an orally consumable outer container enclosing the cannabis. In another embodiment, the capsule is formed as a small, soluble case of gelatin containing the cannabis. In one embodiment, the delivery method includes an oral strip impregnated with an amount of cannabis that can be absorbed by the subject through, for example, the subject's tongue. In another embodiment, the delivery method includes a spray for spraying a liquid form of the cannabis into the subject's mouth for ingestion. In yet another embodiment, the delivery method includes a patch impregnated with a cannabis solution that can be absorbed through the subject's skin. In yet a further embodiment, the cannabis composition 200 can be in the form of a liquid drink. In yet another embodiment, the cannabis can be in the form of a powder that can be mixed with water or other liquid for the subject to consume. In yet another embodiment, the cannabis can be in the form of a tablet, such as a powder compressed tablet. In a preferred embodiment, the cannabis composition 200 is substantially non-odorous. More specifically, the cannabis composition does not emit a substantially detectable amount of cannabis odor so as not to alert any other individuals of the cannabis consumption. These forms can be considered a more discrete and socially acceptable cannabis delivery method, not possessing the negative stigma typically associated with traditional cannabis consumption forms, such as inhalation through smoking and vaporizing, or ingesting edible cannabis food products. As used herein, the term “edible food product” is intended to indicate an orally consumable and ingestible food item that is normally consumed by individuals either as a snack, a drink, or as part of a meal. Providing a discrete and socially acceptable form of cannabis allows subjects to feel free to administer the prescribed cannabis composition 200 in public without the social fear that any potential observers will be able to discern that the subject is consuming cannabis. And, therefore, the subject will not suffer any social drawback for consuming the same. In one embodiment, the method further includes providing dosage guidelines and/or potential side effect warnings in conjunction with the cannabis composition 200. Such guidelines and instruction can be provided on packaging or as a separate paper provided to the subject. The process ends at step 104.

Advantageously, the compositions and methods of the present invention can make cannabis distribution more accessible and acceptable to the general public and provide more medically targeted cannabis compositions tailored specifically to a singular targeted ailment.

What is claimed is:

1. A method of dispensing cannabis, the method comprising:
providing a cannabis composition via a dispensary, the cannabis composition including:
an effective amount of cannabis;
formulated as an ailment-targeted treatment; and
formed so as to deliver the effective amount of cannabis to a subject through a delivery method other than inhalation or consumption of an edible food product.

2. A cannabis composition comprising:
an effective amount of cannabis configured for treatment of a singular specific ailment; and
formed so as to deliver the effective amount of cannabis to a subject through a delivery method other than inhalation or consumption of an edible food product.
